(19) United States
(12) Patent Application Publication
(10) Pub. No.: US 2013/0302385 A1
(43) Pub. Date: Nov. 14, 2013

(54) COMPOSITIONS CONTAINING A WATER-BASED GEL AND A WATER-IN-OIL EMULSION

(71) Applicants: Sara MUENZ, Middlesex, NJ (US); Gisela Perruna, Rahway, NJ (US); Ashini Amin, Monroe, NJ (US)

(72) Inventors: Sara MUENZ, Middlesex, NJ (US); Gisela Perruna, Rahway, NJ (US); Ashini Amin, Monroe, NJ (US)

(73) Assignee: L'Oreal S.A., Paris (FR)

(21) Appl. No.: 13/466,906

(22) Filed: May 8, 2012

(51) Int. Cl. A61K 8/06; A61K 8/91

(52) U.S. Cl. 424/401; 514/772.1; 514/738

(57) ABSTRACT

The invention relates to a composition, especially a cosmetic composition, containing (a) a water-based gel phase; and (b) a water-in-oil emulsion phase.

### FIELD OF THE INVENTION

The present invention generally relates to compositions, for example, cosmetic care, treatment or make-up compositions, comprising (a) a water-based gel phase; and (b) a water-in-oil emulsion phase.

### DISCUSSION OF THE BACKGROUND

Producing stable cosmetic compositions containing both a water-based gel phase and an emulsion phase has been difficult in the past owing to several factors including reactivity of the ingredients in the different phases. Product instability in this type of product manifests itself in various ways such as, for example, the product becoming discolored, malodorous and/or broken down (i.e., the architecture of the composition degrades).

U.S. Pat. No. 6,245,344 discloses dual phase products including a swirl shape in which a first phase is disposed to form a swirl pattern within a second phase. However, this patent does not disclose stable compositions containing both a water-in-oil emulsion and water-based gel. Thus, there is a need for a composition including a water-based gel phase and an emulsion phase which is stable.

Accordingly, one aspect of the present invention is a care and/or makeup and/or treatment composition for keratinous material such as the skin and/or the lips includes a water-based gel phase and an emulsion phase, and which is stable and aesthetically desirable.

### SUMMARY OF THE INVENTION

The present invention relates to compositions, preferably cosmetic compositions, comprising (a) a water-based gel phase; and (b) a water-in-oil emulsion phase. Preferably, the composition is patterned.

The present invention also relates to compositions, preferably cosmetic compositions, comprising (a) a water-based gel phase; and (b) a water-in-silicone emulsion phase. Preferably, the composition is patterned.

The present invention further relates to compositions, preferably cosmetic compositions, comprising (a) a water-based gel phase; (b) a water-in-oil emulsion phase; and (c) at least one water-soluble active agent such as, for example, a humectant or a moisturizing agent.

The present invention further relates to compositions, preferably cosmetic compositions, comprising (a) a water-based gel phase; (b) a water-in-silicone emulsion phase; and (c) at least one water-soluble active agent such as, for example, a humectant or a moisturizing agent.

The present invention also relates to methods of delivering a water-soluble active agent to a keratin material such as skin or lips comprising applying to the keratin material a composition comprising (a) a water-based gel phase; (b) a water-in-oil emulsion phase; and (c) at least one water-soluble active agent such as, for example, a humectant or a moisturizing agent.

The present invention also relates to methods of delivering a water-soluble active agent to a keratin material such as skin or lips comprising applying to the keratin material a composition comprising (a) a water-based gel phase; (b) a water-in-silicone emulsion phase; and (c) at least one water-soluble active agent such as, for example, a humectant or a moisturizing agent.

The present invention further relates to methods of camouflaging and/or disguising skin or lip imperfections comprising applying a skin or lip imperfection camouflaging and/or disguising effective amount of a composition (a) a water-based gel phase; and (b) a water-in-oil emulsion phase to skin or lips in need of such camouflaging or disguising.

The present invention further relates to methods of camouflaging and/or disguising skin or lip imperfections comprising applying a skin or lip imperfection camouflaging and/or disguising effective amount of a composition (a) a water-based gel phase; and (b) a water-in-silicone emulsion phase to skin or lips in need of such camouflaging or disguising.

The present invention also relates to methods of treating, caring for and/or making up keratinous material (for example, skin or lips) by applying compositions of the present invention to the keratinous material in an amount sufficient to treat, care for and/or make up the keratinous material.

The present invention further relates to methods of enhancing the appearance of keratinous material (for example, skin or lips) by applying compositions of the present invention to the keratinous material in an amount sufficient to enhance the appearance of the keratinous material.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

“Transfer resistance” as used herein refers to the quality exhibited by compositions that are not readily removed by contact with another material, such as, for example, a glass, an item of clothing or the skin, for example, when eating or drinking. Transfer resistance may be evaluated by any method known in the art for evaluating such. For example, transfer resistance of a composition may be evaluated by a “kiss” test. The “kiss” test may involve application of the composition to human lips followed by “kissing” a material, for example, a sheet of paper, after expiration of a certain amount of time following application, such as 2 minutes after application. Similarly, transfer resistance of a composition may be evaluated by the amount of product transferred from a wearer to any other substrate, such as transfer from the neck of an individual to a collar after the expiration of a certain amount of time following application. The amount of composition transferred to the substrate (e.g., collar, or paper) may then be evaluated and compared. For example, a composition may be transfer resistant if a majority of the product is left on the wearer, e.g., lips, neck, etc. Further, the amount transferred may be compared with that transferred by other compositions, such as commercially available compositions.

“Long wear” compositions as used herein, refers to compositions where at least one property chosen from consistency, texture, and color remains the same as at the time of application, as viewed by the naked eye, after an extended period of time, such as, for example, 1 hour, 2 hours, and further such as 8 hours. Long wear properties may be evaluated by any method known in the art for evaluating such properties. For example, long wear may be evaluated by a test involving the application of a composition to human skin (including lips) and evaluating the consistency, texture and color of the composition after an extended period of time. For example, the consistency, texture and color of a lip composition may be evaluated immediately following application and these characteristics may then be re-evaluated and compared after an individual has worn the lip composition for a certain amount of time. Further, these characteristics may be evaluated with respect to other compositions, such as commercially available compositions.

“Waterproof” as used herein refers to the ability to repel water and permanence with respect to water. Waterproof properties may be evaluated by any method known in the art for evaluating such properties. For example, a mascara composition may be applied to false eyelashes, which may then be placed in water for a certain amount of time, such as, for example, 20 minutes. Upon expiration of the pre-ascertained amount of time, the false eyelashes may be removed from the water and passed over a material, such as, for example, a sheet of paper. The extent of residue left on the material may then be evaluated and compared with other compositions, such as, for example, commercially available compositions. Similarly, for example, a composition may be applied to skin, and the skin may be submerged in water for a certain amount of time. The amount of composition remaining on the skin after the pre-ascertained amount of time may then be evaluated and compared. For example, a composition may be waterproof if a majority of the product is left on the wearer, e.g., eyelashes, skin, etc.

The cosmetic compositions and methods of the present invention can comprise, consist of, or consist essentially of the essential elements and limitations of the invention described herein, as well as any additional or optional ingredients, components, or limitations described herein or any otherwise useful ingredient found in personal care compositions intended for application to keratin materials.

In accordance with certain aspects of the present invention, the phrase “liquid oil phase” is understood to mean an oil phase, which is liquid at room temperature (25° C.) and atmospheric pressure (760 mmHg), and which comprises one or more fatty substances that are liquid at room temperature, also known as oils, which are compatible with one another.

In accordance with certain aspects of the present invention, the phrase “structured oil phase” is understood to mean that this structured phase does not run between the fingers and/or is at least thickened.

Where the liquid oil phase is structured, it makes it possible to limit exudation of the oil phase from solid compositions, and furthermore, to limit, after deposition on the skin or the lips, its migration into the wrinkles and fine lines, which is desired for compositions such as a lipstick or an eyeshadow. Significant migration of the liquid oil phase, laden with coloring materials, leads to an unaesthetic effect around the lips or the eyes, which can accentuate the wrinkles and fine lines. This migration is often mentioned by women as being a major defect of conventional lipsticks and eyeshadows. The term “migration” is understood to mean running of the composition deposited on the lips or skin beyond its initial outline.

Depending on the intended application, such as a stick, hardness of the composition may also be considered. The hardness of a composition may, for example, be expressed in gramforce (gf). The composition of the present invention may, for example, have a hardness ranging from 20 gf to 2000 gf, such as from 20 gf to 900 gf, and further such as from 20 gf to 600 gf.

This hardness is measured in one of two ways. A first test for hardness is according to a method of penetrating a probe into the composition and in particular using a texture analyzer (for example TA-XT2i from Rheo) equipped with an ebonite cylinder of height 25 mm and diameter 8 mm. The hardness measurement is carried out at 20° C. at the center of 5 samples of the composition. The cylinder is introduced into each sample of composition at a pre-speed of 2 mm/s and then at a speed of 0.5 mm/s and finally at a post-speed of 2 mm/s, the total displacement being 1 mm. The recorded hardness value is that of the maximum peak observed. The measurement error is ±50 gf.

The second test for hardness is the “cheese wire” method, which involves cutting an 8.1 mm or preferably 12.7 mm in diameter stick composition and measuring its hardness at 20° C. using a DFGHS 2 tensile testing machine from Indelco-Chatillon Co. at a speed of 100 mm/minute. The hardness value from this method is expressed in grams as the shear force required to cut a stick under the above conditions. According to this method, the hardness of compositions according to the present invention which may be in stick form may, for example, range from 30 gf to 300 gf, such as from 30 gf to 250 gf, for a sample of 8.1 mm in diameter stick, and further such as from 30 gf to 200 gf, and also further such as from 30 gf to 120 gf for a sample of 12.7 mm in diameter stick.

The skilled artisan may choose to evaluate a composition using at least one of the tests for hardness outlined above based on the application envisaged and the hardness desired. If one obtains an acceptable hardness value, in view of the intended application, from at least one of these hardness tests, the composition falls within preferred embodiments of the invention.

As is evident, the hardness of the composition according to preferred embodiments of the invention may, for example, be such that the composition is advantageously self-supporting and can disintegrate easily to form a satisfactory deposit on the skin and/or the lips and/or superficial body growths, such as keratinous fibers. In addition, this hardness may impart good impact strength to the inventive compositions, which may be molded or cast, for example, in stick or dish form.

“Gloss” is essentially related to the nature of the liquid oil phase. Thus, for example, it is possible to reduce the level of waxes and fillers in a lipstick to increase the gloss of the lipstick, but then the migration of the liquid oil phase increases. In other words, the levels of waxes and/or of fillers necessary for preparation of a stick of suitable hardness have been a restricting factor on the gloss of the deposit.

“Tackiness” as used herein refers to measuring the maximum tensile force, Fₘₐₓ, required while separating two surfaces. Depending on the application envisaged and the formulation being designed, the desirable value for Fₘₐₓ may vary. In some embodiments, the substantially non-tacky compositions have a Fₘₐₓ of less than about 4 Newton (N), less than about 1 N, less than about 0.5 N, less than about 0.3 N, less than about 0.2 N or less than 0.1 N. One of ordinary skill in the art can determine the Fₘₐₓ of the composition by, for example, determining the maximum force of traction, measured with an extensiometer of the LLOYD model LR5K type, needed to detach two surfaces.

For example, two 38 mm² surfaces, A and B, which are solid, rigid, inert, and non-absorbing, are mounted on movable mounts, facing each other. The surfaces may be movable either toward or away from each other, or one may move surface A independently from surface B or vice versa. Prior to insertion into the extensiometer, surface A is coated with the composition to be measured, which may be dissolved in a solvent such as aqueous, hydroalcoholic, hydrocarbon, silicone, and alcoholic solvents in a concentration of from about 10 to about 30%, preferably 20%, the surface A is coated in a thickness of from 1 to 10 mil, preferably 1 mil, and the surface is dried for 24 hours at room temperature, e.g., 22 to 25° C., at a relative humidity of about 50%. Once inserted in the extensiometer, surface A is subjected for 20 seconds to a compression force of 3 N against surface B and then subjected for 30 seconds to tensile force at a rate of 20 mm/minute. The amount of force, Fₘₐₓ, needed to obtain initial separation is then noted. A mean Fₘₐₓ is determined by carrying out the procedure with multiple pairs, preferably at least six pairs, of surface A and surface B.

The composition of the present invention may be in any form. For example, it may be a paste, a solid or a cream. The composition of the invention may be transparent or clear, including for example, a composition without pigments. The composition can also be a molded composition or cast as a stick or a dish. The composition in one embodiment is a solid such as a molded stick or a poured stick. The compositions of the present invention may also be in the form of a lip composition such as a lipstick or a liquid lip color, a foundation or a mascara product.

As defined herein, structural stability can be tested by placing the composition in a controlled environment chamber for 8 weeks at 25° C. In this test, the physical condition of the sample is inspected as it is placed in the chamber. The sample is then inspected again at 24 hours, 3 days, 1 week, 2 weeks, 4 weeks and 8 weeks. At each inspection, the sample is examined for abnormalities in the composition such as phase separation if the composition is in the form of an emulsion, bending or leaning if the composition is in stick form, melting, or syneresis (or sweating). The stability can be further tested by repeating the 8-week test at 40° C., 37° C., 45° C., 50° C. and/or under freeze-thaw conditions. A composition is considered to lack stability if in any of these tests an abnormality that impedes functioning of the composition is observed. The skilled artisan will readily recognize an abnormality that impedes functioning of a composition based on the intended application.

Compositions of the Present Invention

In accordance with the present invention, compositions, preferably cosmetic compositions, containing (a) a water-based gel phase; and (b) a water-in-oil emulsion phase are provided. The compositions of the present invention can comprise, consist essentially of or consist of (a) a water-based gel phase; and (b) a water-in-oil emulsion phase.

According to preferred embodiments, the compositions of the present invention contain an identifiable pattern. According to these preferred embodiments, the pattern can result from the demarcation between the water-based gel phase and the water-in-oil emulsion phase. For example, the water-in-oil emulsion phase can be placed within the water-based gel phase such that the water-in-oil emulsion phase forms an identifiable pattern against the background of the water-based gel phase. Alternatively, the water-based gel phase can be placed within the water-in-oil emulsion such that the water-based gel phase forms an identifiable pattern against the background of the water-in-oil emulsion phase. In accordance with these embodiments, the identifiable pattern can be of any discernable shape, pattern, size. According to most preferred embodiments, the identifiable pattern is a swirl pattern. Also according to most preferred embodiments, the swirl pattern if formed by placing a water-in-oil emulsion phase within a water-based gel phase.

According to preferred embodiments of the present invention, the compositions of the present invention comprise 65% to 75% by weight, more preferably 67% to 73% by weight, and more preferably 69% to 71% by weight of the water-based gel phase with respect to the total weight of the composition.

According to preferred embodiments of the present invention, the compositions of the present invention comprise 25% to 35% by weight, more preferably 27% to 33% by weight, and more preferably 29% to 31% by weight of the water-in-oil emulsion phase with respect to the total weight of the composition.

According to preferred embodiments, the weight ratio of water-based gel phase to water-in-oil emulsion phase is 3:1 to 2:1, more preferably 3.75:1.5 to 3.25:1.5, and more preferably 7:3.

According to particularly preferred embodiments, the compositions of the present invention comprise 67% to 73% by weight of a water-based gel phase and 27% to 33% of a water-in-silicone emulsion phase, wherein the weight ratio of water-based gel phase to water-in-silicone emulsion phase is 3.75:1.5 to 3.25:1.5 and the water-in-silicone emulsion phase is placed in the water-based gel phase in swirl pattern.

Water-Based Gel Phase

According to the present invention, the water-based gel phase comprises water. Preferably, the water present in the water-based gel phase represents from about 50% to about 90% by weight of the weight of the water-based gel phase, more preferably from about 60% to about 90% of the weight of the water-based gel phase, and most preferably from about 70% to about 85% of the weight of the water-based gel phase, including all ranges and subranges therebetween.

According to the present invention, the water-based gel phase comprises at least one film forming agent. According to preferred embodiments, the film forming agent in the water-based gel phase is either liposoluble or water-soluble.

Suitable examples of acceptable film forming agents include, but are not limited to, polyalkylenes, polyvinylpyrrolidone (PVP) or vinylpyrrolidone (VP) homopolymers or copolymers, copolymers of a C2 to C30, such as C3 to C22 alkene, and combinations thereof. As specific examples of VP copolymers which can be used in the invention, mention may be made of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate copolymer. According to particularly preferred embodiments, the film forming agent in the water-based gel phase is polyvinylpyrolidone.

Preferably, the film forming agent represents from about 0.01% to about 1% by weight of the weight of the water-based gel phase, more preferably from about 0.02% to about 0.5% of the weight of the water-based gel phase, and most preferably from about 0.03% to about 0.1% of the water-based gel phase, including all ranges and subranges therebetween. According to most preferred embodiments, the water-based gel phase comprises 0.1% or less of the film forming agent.

According to preferred embodiments of the present invention, the water-based gel phase can further comprise at least one gelling agent in an amount sufficient to form a water-based gel phase. Suitable gelling agents include any agent which is compatible with the water-based gel phase and which is capable of thickening, solidifying or increasing the viscosity of the water-based gel phase. Suitable gelling agents include but are not limited to polysaccharides such as cellulose derivatives (carboxymethylcellulose, hydroxypropylmethylcellulose); natural gums such as xanthan, guar and carob gums, scleroglucans and chitin or chitosan derivatives; proteins or their hydrolysates such as keratin, gelatin and collagen; and acrylic and methacrylic compounds such as acrylate copolymers (including acrylates/C10-C30 alkyl acrylate crosspolymer sold by Lubrizol under the name Carbopol Ultrez 20 polymer), glycerol polyacrylate (including the product sold by the company SEDERMA under the name “LUBRAJEL®”) and ammonium acrylate copolymer (including the product sold by the company HOECHST under the name “PAS 5161®”); polyethylene glycols (PEG) such as the products sold by the company UNION CARBIDE under the name “CARBOWAX®” Also, compounds identified as “Viscosity Increasing Agents—Aqueous” in the International Cosmetic Ingredient Dictionary and Handbook are examples of suitable gelling agents.

Preferably, the gelling agent represents from about 0.1% to about 5% by weight of the weight of the water-based gel phase, more preferably from about 0.3% to about 4% of the weight of the water-based gel phase, and most preferably from about 0.5% to about 3% of the water-based gel phase, including all ranges and subranges therebetween.

According to particularly preferred embodiments of the present invention, the water-based gel phase further comprises at least one water-soluble active agent. Suitable water-soluble active agents include any agent which is compatible with or soluble in water and which is capable of providing a beneficial effect to keratin materials. Examples of suitable water-soluble active agents generally include but are not limited to antioxidants, free-radical scavengers, moisturizers, humectants, bleaching agents, liporegulators, anti-acne agents, antiseborrhoeic agents, anti-ageing agents, softeners, anti-wrinkle agents, keratolytic agents, anti-inflammatories, refreshing agents, cicatrizing agents, vascular protective agents, antibacterials, antifungals, antiperspirants, deodorants, skin conditioners, desensitizing agents, immunomodulators and nourishing agents which are compatible with or soluble in water.

Specific examples of such water-soluble active agents include but are not limited to ascorbic acid and its biologically compatible salts, enzymes, antibiotics, alpha hydroxy acids and their salts, hydroxylated polyacids, sucrose and its derivatives, urea, amino acids, oligopeptides, water-soluble plant and yeast extracts, protein hydrolysates, hyaluronic acid, mucopolysaccharides, vitamins B₂, B₆, H and PP, panthenol, folic acid, acetylsalicylic acid, allantoin, glycyrrhetic acid, kojic acid and hydroquinone.

Particularly preferred water-soluble active agents are humectants or moisturizing agents such as, for example, polyhydroxy compounds including but not limited to glycerin and glycols such as, for example, propylene glycol, butylene glycol, dipropylene glycol and diethylene glycol, glycol ethers such as monopropylene, dipropylene and tripropylene glycol alkyl(C₁-C₄)ethers, monoethylene, diethylene and triethylene glycol.

Preferably, the water-soluble active agent, if present, represents from about 0.1% to about 50% by weight of the weight of the water-based gel phase, more preferably from about 0.5% to about 40% of the weight of the water-based gel phase, more preferably from about 1% to about 30% of the weight of the water-based gel phase, and most preferably from about 3% to about 20% of the water-based gel phase, including all ranges and subranges therebetween.

According to particularly preferred embodiments of the present invention, the water-based gel phase is clear. In accordance with the present invention, “clear” means that the gel phase (excluding insoluble materials, if present in the gel phase) is transparent and/or translucent. More specifically, it means that a layer of the water-based gel phase having a thickness arbitrarily set at 1 cm allows a portion of the visible light to pass through, either while scattering it (bulk translucent compositions) or without scattering it (bulk transparent compositions).

For example, the clear water-based gel phase of preferred embodiments of the present invention can be characterized in one manner in that it has a transmission at a thickness of 10 μm, measured at a wavelength of the maximum of one of the absorption or scattering peaks of a coloring agent in the composition, of between 20% and 80%. Such transmission as defined here is equal to the ratio of the intensity of light transmitted by the sample (Iₜ) to the intensity of light transmitted by the control (Iₒ), express as a %: T(%)=Iₜ/Iₒ. According to such a definition, the preferred sample carrier for determining the transmission of a 10 μm layer is a transparent glass or quartz slide, the size of which depends on the measuring cell of the spectrophotometer used (20 mm×10 mm×3 mm for a Cary 300), exhibiting at its surface a flat recess with a depth of 10 μm. This flat recess is filled with the sample and the excess is optionally leveled down using a slide, so as thus to obtain a perfectly even layer with a thickness of 10 μm. Preferred measurements can be carried out using a double-beam UV/visible spectrophotometer, Cary 300 model from Varian, in transmission mode and by using, as control, a transparent slide (of quartz or of glass) with an identical thickness to that receiving the sample. The transmission values indicated for the water-based gel phase of the compositions of the present invention are those measured at the wavelength corresponding to the maximum of one of the absorption peaks or scattering peaks of a coloring agent in the visible light region (λ=400 to 750 nm).

In accordance with these preferred embodiments, the water-based gel phase contains insufficient colorant, opacifying agent, etc. to render the gel phase unclear (as defined above). According to particularly preferred embodiments, the gel phase contains no colorant and/or opacifying agent.

With respect to stability of the preferred embodiments of the present invention in which the water-based gel is clear, another indication of product stability is lack of discoloration (for example, yellowing) of the water-based gel after 2 weeks, 4 weeks, 2 months and/or 3 months.

Water-in-Oil Emulsion Phase

According to the present invention, compositions comprising a water-in-oil emulsion phase are provided. This emulsion phase contains an external oil phase and an internal water phase. The emulsion can contain droplets of any size. However, it is to be understood that, as with any emulsion, smaller and more homogeneous (that is, less diversity of droplet sizes) droplet sizes are preferred because emulsions containing such droplets tend to be more stable. Any suitable means for preparing emulsions can be used to prepare the water-in-oil emulsion phase of the present invention.

External Oil Phase

According to the present invention, the water-in-oil phase comprises an external oil phase. The external oil phase comprises at least one oil. Any oils can be used in accordance with the present invention. The oils can be volatile or non-volatile, silicone-based and/or hydrocarbon-based, etc. Thus, for example, the external oil phase may contain, independently or in combination, volatile silicone oils, non-volatile silicone oils, volatile non-silicone oils and non-volatile non-silicone oils.

In one embodiment, the compositions of the present invention are substantially free of silicone oils (i.e., contain less than about 1% of silicone oil). In another embodiment, the compositions are substantially free of non-silicone oils (i.e., contain less than about 1% of non-silicone oil). In another embodiment, the compositions are substantially free of non-volatile oils (i.e., contain less than about 1% of non-volatile oil). In yet another embodiment, the compositions are substantially free of volatile oils (i.e., contain less than about 1% of volatile oil).

According to one embodiment, the external oil phase may contain one or more volatile silicone oils. Examples of such volatile silicone oils include linear or cyclic silicone oils having a viscosity at room temperature less than or equal to 6 cSt and having from 2 to 7 silicone atoms, these silicones being optionally substituted with alkyl or alkoxy groups of 1 to 10 carbon atoms. Suitable oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |
| PDMS DC 200 (5 cSt) from Dow Corning | 134 | 5 |
| PDMS DC 200 (3St) from Dow Corning | 102 | 3 |

Further, a volatile linear silicone oil may be employed in the compositions of the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

The volatility of the solvents/oils can be determined using the evaporation speed as set forth in U.S. Pat. No. 6,338,839.

Examples of other silicone oils that may be used in the invention include non-volatile linear polydimethylsiloxanes (PDMSs), that are liquid at room temperature; polydimethylsiloxanes comprising alkyl, alkoxy or phenyl groups, which are pendent and/or at the end of a silicone chain, these groups each containing from 2 to 24 carbon atoms; phenylsilicones, for instance phenyl trimethicones, phenyl dimethicones, phenyl trimethylsiloxydiphenylsiloxanes, diphenyl dimethicones, diphenyl methyldiphenyl trisiloxanes and 2-phenylethyl trimethylsiloxysilicates.

The external oil phase may contain one or more non-silicone volatile oils and may be selected from volatile hydrocarbon oils, alcohols, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, isohexadecane, and for example, the oils sold under the trade names of Isopar or Permethyl, the C₈ to C₁₆ branched esters such as isohexyl or isodecyl neopentanoate and their mixtures. Preferably, the volatile non-silicone oils have a flash point of at least 40° C.

TABLE 2

| Compound | Flash Point (° C.) |
|---|---|
| Isododecane | 43 |
| Isohexadecane | 102 |
| Isodecyl Neopentanoate | 118 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

Examples of other non-silicone oils which can be used in the compositions of the present invention include polar oils such as:
hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;
synthetic oils or esters of formula R₅COOR₆ in which R₅ represents a linear or branched higher fatty acid residue containing from 1 to 40 carbon atoms, including and better still from 7 to 19 carbon atoms, and R₆ represents a branched hydrocarbon-based chain containing from 1 to 40 carbon atoms, including and better still from 3 to 20 carbon atoms, with R₆+R₇≦10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;
synthetic ethers containing from 10 to 40 carbon atoms;
C₆ to C₂₆ fatty alcohols, for instance oleyl alcohol; and
mixtures thereof.

According to particularly preferred embodiments, the external oil phase is a silicone oil phase. That is, the external oil phase comprises primarily silicone oils. For example, the external oil phase preferably contains 60% by weight silicone oil(s) with respect to the weight of total oils in the external oil phase, more preferably 70%, more preferably 80% and more preferably 90% by weight silicone oil(s) with respect to the weight of total oils in the external oil phase. In such embodiments, the emulsion phase can be referred to as a water-in-silicone emulsion phase.

Preferably, the oils present in the external oil phase represent from about 5% to about 70% by weight of the total weight of the water-in-oil phase, more preferably from about 10% to about 60% of the total weight of the composition, and most preferably from about 15% to about 50%, including all ranges and subranges therebetween.

According to the present invention, the water-in-oil emulsion phase comprises at least one polyurethane/poly(meth)acrylate graft copolymer. Preferably, the at least one polyurethane/poly(meth)acrylate graft copolymer is in the external oil phase of the water-in-oil emulsion phase. However, the at least one polyurethane/poly(meth)acrylate graft copolymer can be in the internal water phase of the water-in-oil emulsion phase. Suitable polyurethane/poly(meth)acrylate graft copolymers include but are not limited to those disclosed in U.S. patent application publication no. 2004-0136937, the entire contents of which is hereby incorporated by reference.

The polyurethane/poly(meth)acrylate graft copolymers may also be referred to as an interpenetrated polymer network (IPN) of a polyurethane and a poly(meth)acrylate. As used herein, the expression “interpenetrated polymer network” refers to a blend of two interlaced polymers, obtained by simultaneous polymerization and/or crosslinking of two types of monomer, the blend obtained having a single glass transition temperature.

Preferred IPNs include those which are commercially available from the company Air Products under the name Hybridur. An IPN that is particularly preferred is in the form of an aqueous dispersion of particles e.g., with a weight-average size of between 90 and 110 nm and a number-average size of about 80 nm. This IPN preferably has a glass transition temperature, Tg, ranging from about −60° C. to +100° C. An IPN of this type is available from Air Products under the trade name Hybridur 875 (INCI name: POLYURETHANE-2 (and) POLYMETHYL METHACRYLATE)(which contains approximately 58% water). Polyurethane/poly(meth)acrylics available from Air Products under the names Hybridur X-01602 and X 18693-21 are also preferred.

Preferred IPNs such as those discussed above are disclosed in U.S. patent application publication nos. 2003/0215476, 2004/0136937, 2005/0249763, the entire contents of all of which is hereby incorporated by reference.

According to preferred embodiments, the IPNs are polyurethane/poly(meth)acrylate graft copolymers having the following general structure:

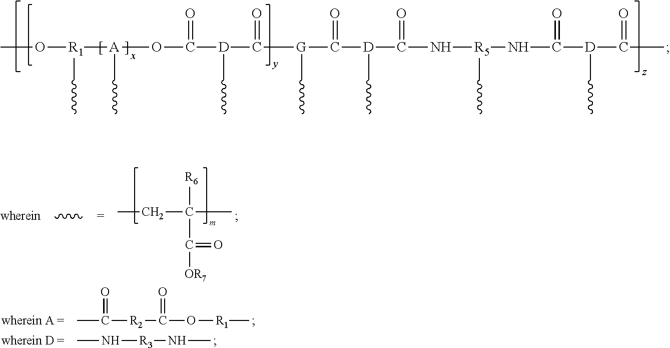

and wherein

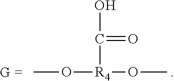

In the formula, R₁, R₂, R₃, R₄, R₅ and R₇ each independently represents an aliphatic hydrocarbon; m represents zero or a positive integer; R₆ represents hydrogen or methyl; and x, y and z each independently represents a positive integer. The graft copolymers may be provided in the form of aqueous dispersions. The graft copolymers may be added to the other components of the composition in powdered form as well.

Generally speaking, preferred ranges of polyurethane/poly(meth)acrylate graft copolymer active material in the water-in-oil emulsion phase are from about 0.1% to about 50% by weight of the weight of the emulsion phase, more preferably from about 0.5% to about 40% of the weight of the emulsion phase, more preferably from about 1% to about 20% of the weight of the emulsion phase, and more preferably from about 2% to about 8% of the weight of the emulsion phase, including all ranges and subranges therebetween.

According to preferred embodiments of the present invention, the water phase of the water-in-oil emulsion phase comprises at least one water-soluble active agent. Suitable water-soluble active agents and the amounts thereof in the water phase of the emulsion phase are the same as discussed above in connection with the water-based gel phase.

According to preferred embodiments of the present invention, the water-in-oil emulsion phase further comprises at least one coloring agent. Suitable coloring agents include but are not limited to pigments, dyes, such as liposoluble dyes, nacreous pigments, and pearling agents.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow. The liposoluble dyes, when present, generally have a concentration ranging up to 20% by weight of the total weight of the composition, such as from 0.0001% to 6%.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, be present in the composition in a concentration ranging up to 50% by weight of the total weight of the composition, such as from 0.1% to 20%, preferably from 0.1% to 15%.

The pigments, which may be used according to the present invention, may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the pigments may be present in the composition in a concentration ranging up to 50% by weight of the total weight of the composition, such as from 0.5% to 40%, and further such as from 2% to 30%. In the case of certain products, the pigments, including nacreous pigments, may, for example, represent up to 50% by weight of the composition.

### Additional Additives

The compositions of the present invention can also comprise any additive usually used in the field under consideration. For example, non-encapsulated pigments, film forming agents, dispersants, antioxidants, essential oils, preserving agents, fragrances, liposoluble polymers that are dispersible in the medium, fillers, neutralizing agents, silicone elastomers, cosmetic and dermatological oil-soluble active agents such as, for example, emollients, moisturizers, vitamins, anti-wrinkle agents, essential fatty acids, sunscreens, and mixtures thereof can be added. A non-exhaustive listing of such ingredients can be found in U.S. Pat. No. 7,879,316, filed Dec. 12, 2003, the entire contents of which is hereby incorporated by reference. Further examples of suitable additional components can be found in the other references which have been incorporated by reference in this application, including but not limited to the applications from which this application claims priority. Still further examples of such additional ingredients may be found in the International Cosmetic Ingredient Dictionary and Handbook (9^{th} ed. 2002).

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present).

Needless to say, the composition of the invention should be cosmetically or dermatologically acceptable, i.e., it should contain a non-toxic physiologically acceptable medium and should be able to be applied to the keratin material (such as skin, superficial body growths or the lips) of human beings. For the purposes of the invention, the expression “cosmetically acceptable” means a composition of pleasant appearance, odor, feel and/or taste.

According to a preferred embodiment of the present invention, the water-in-oil emulsion phase comprises at least one silicone elastomer, wherein the elastomer is present in an amount of at least 1% by weight with respect to the total weight of the composition, more preferably at least 5% by weight, more preferably at least 10% by weight with respect to the total weight of the composition.

According to preferred embodiments of the present invention, methods of treating, caring for and/or making up keratinous material such as skin, lips, hair and mucous membranes by applying compositions of the present invention to the keratinous material in an amount sufficient to treat, care for and/or make up the keratinous material are provided.

According to other preferred embodiments, methods of covering or hiding defects associated with keratinous material such as imperfections or discolorations by applying compositions of the present invention to the keratinous material in an amount sufficient to cover or hide such defects are provided.

According to yet other preferred embodiments, methods of enhancing the appearance of keratinous material by applying compositions of the present invention to the keratinous material in an amount sufficient to enhance the appearance of the keratinous material are provided.

According to preferred embodiments of the present invention, methods for delivering an active agent to keratin materials such as skin or lips comprising applying to the keratin material a composition comprising (a) a water-based gel phase; (b) a water-in-oil emulsion phase; and (c) an active agent are provided.

In accordance with the preceding preferred embodiments, the compositions of the present invention are applied topically to the desired area of the skin in an amount sufficient to treat, care for and/or make up the keratinous material, to cover or hide defects associated with keratinous material, skin imperfections or discolorations, or to enhance the appearance of keratinous material. The compositions may be applied to the desired area as needed, preferably once or twice daily, more preferably once daily and then preferably allowed to dry before subjecting to contact such as with clothing or other objects. The composition is preferably applied to the desired area that is dry or has been dried prior to application. The compositions of the present invention make it possible to obtain superior consumer aesthetics without compromising stability. The preferred ratios and weight percentages identified above provide sufficient medium coverage of product without being perceived as cakey, and provide a nice smoothing/evening effect of the skin. They also provide a pleasant fresh feel on the skin upon application of the composition.

The present invention also envisages kits and/or prepackaged materials suitable for consumer use containing one or more compositions according to the description herein. The packaging and application device for any subject of the invention may be chosen and manufactured by persons skilled in the art on the basis of their general knowledge, and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

The following water-in-silicone emulsion phase(s) and water based gel phase(s) can be combined in accordance with the disclosure in U.S. Pat. Nos. 6,516,838, 6,213,166, and 6,367,519, the entire contents of all of which are hereby incorporated by reference in their entirety.

### Example 1

### Foundation

Water in Silicone Emulsion Phase

The following water-in-silicone emulsion phase was prepared.

| Phase | INCI Name | % |
|---|---|---|
| A1 | Isododecane | 3.775 |
| | Dimethicone | 3.000 |
| | Ethylhexyl | 7.500 |
| | Methoxycinnamate | |
| | Isononyl Isononanoate | 3.050 |
| | PEG-9 | 2.000 |
| | polydimethylsiloxyethyl | |
| | dimethicone | |
| | Disteardimonium | 2.000 |
| | Hectorite | |
| | Polyurethane-2 (and) | 10.000 |
| | Polymethyl Methacrylate | |
| A2 | Titanium Dioxide (and) | 13.540 |
| | Disodium Stearoyl | |
| | Glutamate (and) | |
| | Aluminum Hydroxide | |
| | Iron Oxides (and) | 0.930 |
| | Disodium Stearoyl | |
| | Glutamate (and) | |
| | Aluminum Hydroxide | |
| | Iron Oxides (and) | 0.450 |
| | Disodium Stearoyl | |
| | Glutamate (and) | |
| | Aluminum Hydroxide | |
| | Iron Oxides (and) | 0.080 |
| | Disodium Stearoyl | |
| | Glutamate (and) | |
| | Aluminum Hydroxide | |
| A3 | Acrylates Copolymer | 0.200 |
| | Tocopherol | 0.125 |
| | Ascorbyl Palmitate | 0.125 |
| | Menthyl Lactate | 2.000 |
| A4 | Dimethicone (and) | 34.000 |
| | Dimethicone | |
| | Crosspolymer | |
| | Mica (and) Titanium | 0.375 |
| | Dioxide | |
| | Mica (and) Titanium | 0.125 |
| | Dioxide | |
| B | Water | 7.000 |
| | GLYCERIN | 5.000 |
| | Chlorphenesin | 0.200 |
| | Citric Acid | 0.05 |
| | Potassium Sorbate | 0.1 |
| | Sodium Dehydroacetate | 0.2 |
| | Ethylhexylglycerin | 0.3 |
| | Phenoxyethanol | 0.8 |
| | Panthenol | 0.125 |
| | Lycium Barbarum Fruit | 0.200 |
| | Extract | |
| | Glutamylamidoethyl | 0.250 |
| | Indole | |
| | Acetyl Hexapeptide-1 | 0.250 |
| | (and) Dextran | |
| | Carnosine | 0.250 |
| C | Mica | 2.000 |

This water-in-silicone emulsion phase was prepared by mixing the components of the A1 phase in a main kettle at room temperature for approximately 5 minutes. The temperature was maintained at 25-30° C., and then the components of phase A2 were added to the kettle and mixed for approximately 45 minutes until uniform. Then, phase A3 was added to the kettle and mixed for approximately 10 minutes while continuing to maintain the temperature at 25-30° C. Then, phase A4 was added to the kettle and mixed for approximately 5 minutes at 25-30° C. Next, phase B was added to the kettle slowly over a period of about 5 minutes and mixed for approximately 15 minutes, followed by adding phase C to the kettle at 25-30° C. and mixed until uniform for 10 minutes.

Water Based Gel Phase

The following water-based gel phase was prepared.

| Phase | INCI Name | % |
|---|---|---|
| A1 | Deionized Water | 79.150 |
| | Acrylates/C10-30 Alkyl Acrylate | 0.750 |
| | Crosspolymer | |
| | Glycerin | 15.000 |
| | Disodium EDTA | 0.100 |
| A2 | Phenoxyethanol | 0.500 |
| | Denatured Alcohol | 4.000 |
| B | Sodium Hydroxide | 0.315 |
| C | PVP | 0.085 |
| D | PEG-12 Dimethicone | 0.100 |
| | TOTAL = | 100.000 |

This water-based gel phase was prepared by heating the components of the A1 phase in a main kettle to 40° C. and mixing with a U-blade until uniform. The temperature was then dropped to 25° C., and then the components of phase A2 were added to the kettle and mixed until uniform. Then, phase B was added to the kettle and mixed for 10-15 minutes until uniform. Next, phase C was added and mixed until uniform after which phase D was added and mixed until uniform. Then, the composition was mixed slowly for 15 minutes to reduce air bubbles.

### Example 2

### Water-Based Gel Phase Employing Different Manufacturing Process

The following composition was prepared by the following process.

| Phase | INCI Name | % |
|---|---|---|
| A1 | Deionized Water | 72.315 |
| | PVP | 0.085 |
| A2 | Acrylates/C10-30 Alkyl Acrylate | 0.750 |
| | Crosspolymer | |
| A3 | Deionized Water | 6.52 |
| | Glycerin | 15.000 |
| | Disodium EDTA | 0.100 |
| | Phenoxyethanol | 0.500 |
| | PEG-12 Dimethicone | 0.100 |
| A4 | Deionized Water | 0.315 |
| | Sodium Hydroxide | 0.315 |
| A5 | Denatured Alcohol | 4.000 |
| | TOTAL = | 100.000 |

This water-based gel phase was also prepared by adding the components of the A1 phase in a main kettle and mixing until uniform (about 5 minutes). Temperature was maintained at 25° C. to 30° C. Then, phase A2 was added to the kettle and mixed until uniform (about 35 minutes). Next, the components of phase A3 were added and mixed for 10 minutes. Then, phase A4 was pre-mixed in a side kettle until uniform and subsequently added to the main kettle slowly and mixed for 10 minutes. Then, phase A5 was added and mixed for 10 minutes.

### Example 3

### Further Examples of Water-Based Gel Phases

The following water-based gel phases were prepared as described in the first procedure in Example 1.

Water-Based Gel Phase A

| Phase | INCI Name | % |
|---|---|---|
| A1 | Deionized Water | 76.840 |
| | Acrylates/C10-30 Alkyl Acrylate | 0.450 |
| | Crosspolymer | |
| | Glycerin | 5.000 |
| | Disodium EDTA | 0.100 |
| A2 | Phenoxyethanol | 0.500 |
| | Denatured Alcohol | 15.000 |
| B | Sodium Hydroxide Soln. (50% | 0.110 |
| | aqueous) | |
| C | PVP | 2.000 |
| | TOTAL = | 100.000 |

Water-Based Gel Phase B

| Phase | INCI Name | % |
|---|---|---|
| A1 | Deionized Water | 83.885 |
| | Acrylates/C10-30 Alkyl Acrylate | 0.750 |
| | Crosspolymer | |
| | Glycerin | 7.000 |
| | Disodium EDTA | 0.100 |
| A2 | Phenoxyethanol | 0.500 |
| | Denatured Alcohol | 7.5000 |
| B | Sodium Hydroxide Soln. (50% | 0.180 |
| | aqueous) | |
| C | PVP | 0.085 |
| | TOTAL = | 100.000 |

### Example 4

### Further Water-in-Silicone Emulsion Phase

The following water-in-silicone emulsion phase was prepared as described in Example 1.

| Phase | INCI Name | % |
|---|---|---|
| A1 | Isododecane | 4.200 |
| | Dimethicone | 7.000 |
| | Isononyl Isononanoate | 8.850 |
| | PEG-9 | 2.000 |
| | polydimethylsiloxyethyl | |
| | dimethicone | |
| | Disteardimonium | 1.500 |
| | Hectorite | |
| A2 | Titanium Dioxide (and) | 15.000 |
| | Disodium Stearoyl | |
| | Glutamate (and) | |
| | Aluminum Hydroxide | |
| | (White, Yellow, Red, | |
| | Black) | |
| A3 | Acrylates Copolymer | 0.200 |
| | Vitamin EI | 0.050 |
| | Vitamin C | 0.050 |
| | Menthyl Lactate | 2.000 |
| A4 | Dimethicone (and) | 32.000 |
| | Dimethicone | |
| | Crosspolymer | |
| | Mica (and) Silica | 0.500 |
| B | Water | 7.750 |
| | GLYCERIN | 5.000 |
| | Chlorphenesin | 0.200 |
| | Citric Acid | 0.050 |
| | Potassium Sorbate | 0.100 |
| | Sodium Dehydroacetate | 0.200 |
| | Ethylhexylglycerin | 0.300 |
| | Phenoxyethanol | 0.800 |
| | Panthenol | 0.050 |
| | Lycium Barbarum Fruit | 0.200 |
| | Extract | |
| | Glutamylamidoethyl | 0.250 |
| | Indole | |
| C | Mica | 2.000 |

What is claimed is:

1. A composition comprising (a) a water-based gel phase comprising water and at least one film forming agent; and (b) a water-in-oil emulsion phase comprising at least one oil and at least one polyurethane/poly(meth)acrylate graft copolymer.

2. The composition according to claim 1, wherein the film forming agent is a water-soluble film forming agent.

3. The composition according to claim 1, wherein the film forming agent is a vinylpyrrolidone homopolymer or copolymer.

4. The composition according to claim 1, wherein the film forming agent is a vinylpyrrolidone homopolymer.

5. The composition according to claim 1, wherein the film forming agent is present in an amount of 0.1% or less by weight with respect to the weight of the water-based gel phase.

6. The composition according to claim 4, wherein the film forming agent is present in an amount of 0.1% or less by weight with respect to the weight of the water-based gel phase.

7. The composition according to claim 1, wherein the composition comprises 65% to 75% by weight of the water-based gel phase with respect to the total weight of the composition.

8. The composition according to claim 1, wherein the composition comprises 25% to 35% by weight of the water-in-oil emulsion phase with respect to the total weight of the composition.

9. The composition according to claim 1, wherein the weight ratio of water-based gel phase to water-in-oil emulsion phase is 3:1 to 2:1.

10. The composition according to claim 1, wherein the water-based gel phase comprises at least one gelling agent.

11. The composition according to claim 1, wherein the water-based gel phase comprises at least one water-soluble active agent.

12. The composition according to claim 11, wherein the water-soluble active agent is a humectant or moisturizing agent.

13. The composition according to claim 11, wherein the water-soluble active agent is glycerin or a glycol.

14. The composition of claim 1, wherein the polyurethane/poly(meth)acrylate graft copolymer is present as an aqueous dispersion of particles.

15. The composition of claim 14, wherein the particles have a weight-average size of between about 90 nm and about 110 nm, a number-average size of about 80 nm, and a glass transition temperature ranging from about −60° C. to about +100° C.

16. The composition of claim 1, wherein the polyurethane/poly(meth)acrylate graft copolymer is present in the composition in an amount of 1% to about 20% by weight relative to the total weight of the composition.

17. The composition of claim 1, wherein the water-in-oil emulsion phase comprises at least one colorant.

18. The composition of claim 1, wherein the water-in-oil emulsion phase comprises at least 5% silicone elastomer.

19. A composition comprising (a) a water-based gel phase comprising water and at least one film forming agent; and (b) a water-in-silicone emulsion phase comprising at least one oil and at least one and at least one polyurethane/poly(meth)acrylate graft copolymer, wherein the composition comprises 67% to 73% by weight of the water-based gel phase and 27% to 33% of the water-in-silicone emulsion phase, wherein the weight ratio of water-based gel phase to water-in-silicone emulsion phase is 3.75:1.5 to 3.25:1.5, and wherein the water-in-silicone emulsion phase is placed in the water-based gel phase in swirl pattern.
